(19) United States
(12) Patent Application Publication
(10) Pub. No.: US 2009/0186054 A1
(43) Pub. Date: Jul. 23, 2009

(54) COMPOSITION WITH CONTROLLABLE APPEARANCE

(75) Inventors: Wilhelmus Franciscus Johannes Fontijn, Eindhoven (NL); Evert Jan Van Loenen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/301,313

(22) PCT Filed: May 11, 2007
(86) PCT No.: PCT/IB2007/051792

§ 371 Date: Nov. 18, 2008

(30) Foreign Application Priority Data
May 23, 2006 (EP) 06114423.4

(51) Int. Cl. A61K 8/02; B05C 1/00; B05C 11/00; A45D 40/26

(52) U.S. Cl. 424/401; 118/200; 118/708; 132/320

(57) ABSTRACT

A composition intended to be applied on a surface and having an appearance controllable by an external field is disclosed. The composition comprises: a plurality of field responsive particles with a particle appearance, and a liquid with a liquid appearance, the liquid appearance being distinct from the particle appearance, the liquid being disposed to fixate the plurality of field responsive particles, the composition being disposed such that if the external field is applied to the composition, the field responsive particles are affected with a force being distinct from surrounding liquid.

The present invention relates to compositions with controllable appearance, and more particularly to compositions with an appearance controllable by an external field.

Self-expression is becoming more and more popular. Not only are more ways of self-expression being explored, but also ways to make a specific expression more personal. One of the related areas where a lot of money and marketing power is concentrated is the beauty and make-up industry.

Most make-up known in the art is monochromatic. Some make-up products have a combination of colours that are evenly distributed, or that shift with an angle of inspection. However, to produce a fine pattern, one or more products have to be carefully applied in the desired pattern, requiring a significant amount of work and time.

WO 2005/046576 discloses a tattoo ink composition with magnetic particles. While this method allows to be moved by a magnetic field, the particles still have to by inserted into the skin, using conventional tattoo methods.

Consequently, there is a need to provide a simple and safe way to control the appearance of compositions that can be applied to objects such as a person.

In view of the above, an objective of the invention is to solve or at least reduce the problems discussed above.

Generally, the above objectives are achieved by the attached independent patent claims. A first aspect of the invention is a composition intended to be applied on a surface, the composition having appearance controllable by an external field, the composition comprising a plurality of field responsive particles with a particle appearance, and a liquid with a liquid appearance, the liquid appearance being distinct from the particle appearance, the liquid being disposed to fixate the plurality of field responsive particles, the composition being disposed such that if the external field is applied to the composition, the field responsive particles are affected with a force being distinct from surrounding liquid. With such a composition, it is possible to create fine patterns on sensitive surfaces. It is easy and safe to apply the external field and provides a great deal of control of the appearance.

The surface may be a surface of a person. The composition can then be used as a make-up with great possibilities of changing appearance according to preferences.

The surface may be a surface of an animal. This could be useful for marking animals.

The field responsive particles may be electrically charged and the external field may be an electrical field.

The field responsive particles may be magnetically responsive and the external field may be a magnetic field.

The liquid may be disposed to fixate the plurality of field responsive particles through evaporation. This allows common liquids such as water or organic solvents to be used.

The liquid may comprise monomers and the liquid may be disposed to fixate the plurality of field responsive particles through polymerisation. This allows a creation of thin foils that carry the image without the support of another surface. In other words, the foils could be moved after the pattern is fixated.

The liquid may be disposed to initiate fixation of the plurality of field responsive particles through an action selected from a group of actions comprising heat application, chemical reaction and ultraviolet light application.

The composition may be a paint, allowing a persistent image to be applied.

The composition may be a crème, the crème being removable after having been applied to a person, allowing a temporary image to be applied.

A second aspect of the invention is a pattern generator for applying a field in accordance with a desired pattern to control appearance of a composition according to the first aspect, the pattern generator selected from a group comprising a field pen, a field roller and a field plate. These pattern generators allow points lines and matrixes to be created in the composition, making up a desired image.

The pattern generator may additionally be configured to apply the composition on a person. This allows the composition to be used as make-up.

The plurality of fields may be arranged in a matrix corresponding to pixels of an image. This enables the image to be controlled by an external controller, e.g. a computer to create an image, e.g. a photo, stored in the computer.

A third aspect of the invention is an applicator for applying a composition according to the first aspect on a person, wherein a surface of the applicator for application of the composition is hydrophobic and the composition is hydrophilic, or the other way around. This prevents the composition from sticking to the applicator.

Other objectives, features and advantages of the present invention will appear from the following detailed disclosure, from the attached dependent claims as well as from the drawings.

Generally, all terms used in the claims are to be interpreted according to their ordinary meaning in the technical field, unless explicitly defined otherwise herein. All references to “a/an/the element, device, component, means, step, etc” are to be interpreted openly as referring to at least one instance of the element, device, component, means, step, etc., unless explicitly stated otherwise. The steps of any method disclosed herein do not have to be performed in the exact order disclosed, unless explicitly stated.

Embodiments of the present invention will now be described in more detail, reference being made to the enclosed drawings, in which:

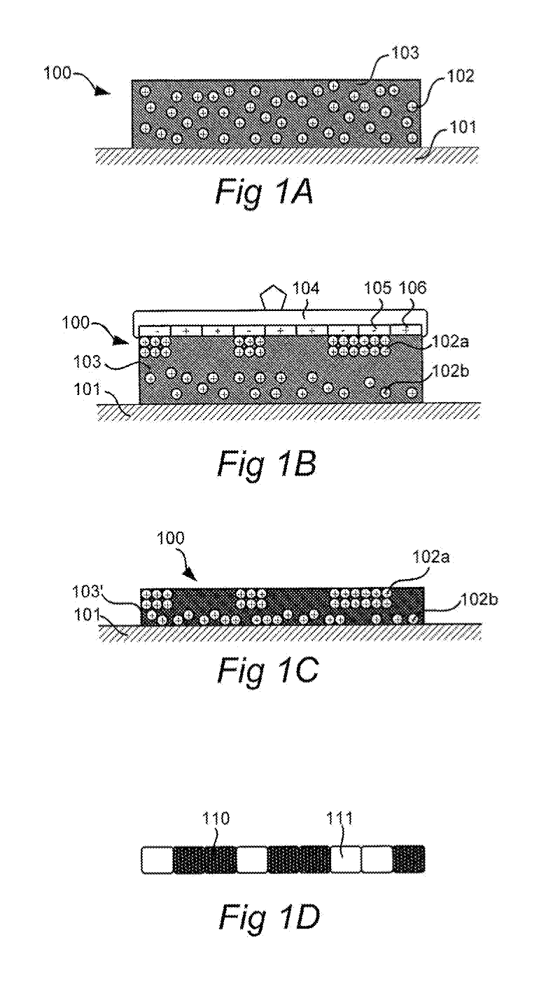

FIGS. 1A-D are schematic diagrams of an embodiment of the present invention where electric force is utilised.

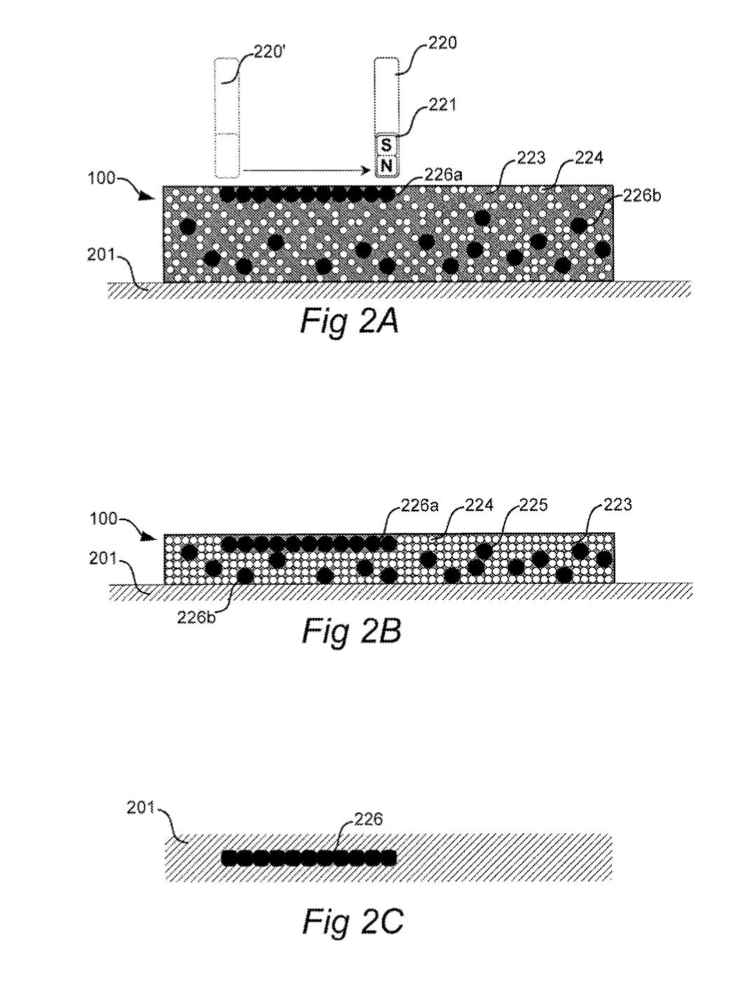

FIGS. 2A-C are schematic diagrams of an embodiment of the present invention where magnetic force is utilised.

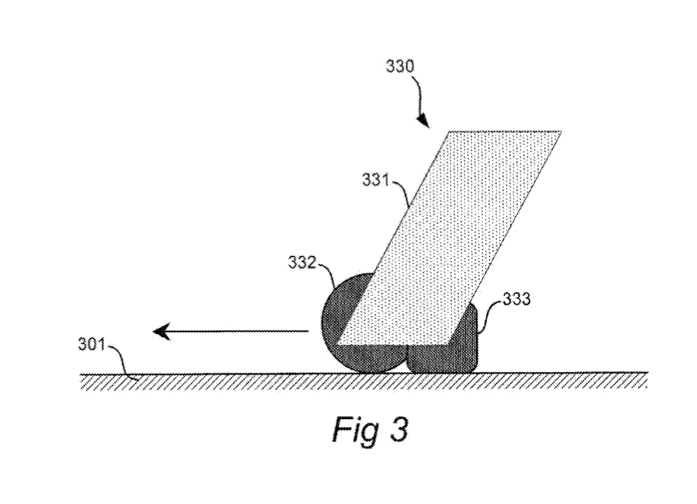

FIG. 3 is a schematic diagram of a combined applicator and pattern generator device 330 in an embodiment of the present invention.

FIGS. 1A-D show an embodiment of the present invention where electric force is utilised. In FIG. 1A, a composition 100 has been applied to a surface 101, such as human skin. The composition 100 comprises particles 102 surrounded by a liquid 103. The particles 102, may for example comprise copper chromite. The particles are electrically charged, in this example positively charged, and have an appearance that differs from surrounding liquid, such as a different colour. The liquid 103 is either not charged or consists of particles of an opposite charge and/or different colour compared to the particles 102. As the suspension is electrically neutral, either there are particles or ions of an opposite charge in the liquid.

The composition is applied to the surface with an applicator, where in one embodiment, the composition is hydrophilic and the applicator is hydrophobic (or vice versa), preventing composition from sticking to the applicator.

In FIG. 1B, a stamp 104 comprising negatively charged areas 105 and positively charged areas 106 is applied to the composition 100. The different charges of the stamp 104 are arranged in any desired pattern, where the pattern of the charges will eventually result in a similar pattern on the skin 101. As can be seen in FIG. 1B, the differently charged areas are only seen from a side view. In reality, a two dimensional matrix can be useful, having the ability to create two-dimensional images on the surface 101. The matrix then comprises a number of rows, where the charged areas 105, 106 make up one such row. Each charged area then corresponds to a pixel in a two dimensional image. The stamp 104 is connected to a controller (not shown) and memory (not shown), the controller and the memory being located either within the stamp 104 itself or externally, connected to the stamp 104 by a cable or through a wireless connection. The controller controls the charge of each charged area 105,106 to correspond to a pixel in the desired image. Note that switching between two states (positive/negative) on one pixel can produce grey values. Alternatively, a zero-dimensional pen (which gives a line if moved), or a one-dimensional brush (which gives an area if moved), can be used in stead of the stamp to render a desired picture.

When the stamp is applied, electrical forces affect the particles 102. As the particles 102 are positively charged in this example, there is an attractive force towards negatively charged areas 105 of the stamp, while there is a repulsive force away from the positive areas 106 of the stamp 104. Therefore, close to the stamp 104, particles 102 are attracted to negative areas 105 and repulsed from the positive areas 106, effectively creating a pattern of particles 102a corresponding to the stamp. Some particles 102b may be too deep in the liquid 103, where the liquid 103 has too much viscosity for the electric force to have any significant effect on the movement of these particles 102b, or the particles 102b are too much shielded from the stamp due to the negatively charged particles/ions.

After the particles 102a have been arranged in the desired pattern according to the stamp 104, the stamp 104 is removed and the composition is fixated, to a state as can be seen in FIG. 1C. Fixation implies that the relative position of the particles 102 is fixed and is not easily changed, thereby also fixating the desired pattern. For example, using evaporation, the liquid 103 evaporates and fixates the particles 102. Fixation could also be achieved by a chemical reaction, for instance due to exposure to air (i.e. oxygen), by applying heat or UV light. Another particularly attractive way to fixate is the use of monomers in the liquid 103 that polymerise during fixation, as this results in foils which could carry the image without the support of another surface. The charged particles 102 can also be embedded in (or even connected to) a polymer matrix that is kept flexible by the liquid 103 and that sets as soon as the liquid 103 evaporates.

For evaporation, the liquid can be any solution used for paint including water and organic solvents e.g. ethanol. For chemical reaction the liquid can for example be epoxy resin. For polymerisation, the liquid can for example be methyl methacrylate (MMA).

It is to be noted that the relative dimensions in FIGS. 1A-D are exaggerated for ease of understanding. For example, the particles 102 are much smaller, the number of particles 102 and charged areas on the stamp 104 are typically much higher, the stamp would be much thicker, etc.

FIGS. 2A-C show an embodiment of the present invention where magnetic force is utilised. In FIG. 2A, a composition 200 has been applied to a surface 201, such as human skin. The composition 200 comprises magnetic particles 226a-b surrounded by a liquid 223. The magnetic particles 226a-b are magnetically responsive, implying that the magnetic particles 226a-b are responsive to magnetic fields. The magnetic particles 226a-b may for example comprise ferrite or Permalloy. Furthermore, the magnetic particles 226a-b have an appearance that differs from the surrounding liquid 223, such as a different colour. Optionally, the liquid 223 can comprise neutral particles 224 which are significantly less magnetically responsive than the magnetic particles 226-b, where the neutral particles are of a different colour compared to the magnetic particles 226a-b. The liquid 223 itself is also significantly less responsive to magnetic fields than the magnetic particles 226a-b.

As with the embodiment where electrical force is utilised, the composition is applied to the surface 201 with an applicator, where in one embodiment, the composition is hydrophilic and the applicator is hydrophobic (or vice versa), preventing composition from sticking to the applicator.

A pattern generator 220 comprises a magnet 221. When the magnet 221 is positioned close to the composition 200, it will result in an attractive force on the magnetic particles 226a-b. If the magnet then is moved along the surface of the composition, e.g. from position 221′ to 221, the magnetic particles 226a will move close to the position of the magnet along the line thus formed. The magnetic particles 226b have an inertia that counteracts the magnetic force applied on them, whereby these particles 226b essentially stay in the same position as before. As the magnetic particles 226a are of a different colour to the surrounding liquid 223 and any neutral particles 224, this line will be visible.

Instead of using a moving magnet to create a desired pattern, electromagnets which are more easily controllable, can be used. The electromagnets can for example be arranged in a matrix forming pixels that make up a desired image.

Fixation then occurs, using one of the processes described above in conjunction with FIG. 1B, resulting in a state as can be seen in FIG. 2B.

FIG. 2C shows a view from above of the line formed on the surface 201.

FIG. 3 schematically shows a combined applicator and pattern generator device 330 in an embodiment of the present invention. The device 330 can be used with the composition being responsive to electric fields, the composition being responsive to magnetic fields, or it can be configurable to be able to be used with both these types of composition. The device 330 comprises a handle 331 allowing a person to grip and move the device. A roller 332 works as an applicator, applying an even distribution of composition with particles (magnetic or electrically charged) on a surface 301. A pattern generator 333 applies electric charges (in the case of electrically charged particles) or magnetic fields (in the case of magnetic particles) to the composition to create a pattern of the particles in the composition. The pattern generated by the pattern generator 333 is controlled by a controller (not shown) and a memory (not shown), which may also be comprised in the device.

The roller 332 measures the speed of the whole device 330 across the surface, sends this information to the controller, which then can adjust the write speed accordingly, allowing any pattern to be effected on the surface, only restricted by pixel size of the pattern generator. For example, the device 330 can be used to easily create photographic images on human skin.

If the device 330 is wide enough for the image to be created, only one roll is necessary. Otherwise several rolls with the device 330 can create an image arbitrarily wide, on a row per row basis.

The roll can also be used to apply heat, or to apply UV from a lamp in the core of the roller.

The invention has mainly been described above with reference to a few embodiments. However, as is readily appreciated by a person skilled in the art, other embodiments than the ones disclosed above are equally possible within the scope of the invention, as defined by the appended patent claims.

1. A film forming composition (100, 200) comprising a fluid matrix (103, 223) and a plurality of particles (102a, 226a) dispersed in the fluid matrix (103, 223), suitable for application on a surface, wherein
the plurality of particles (102a, 226a) are field responsive such that if an external field is applied to said composition (100, 200) when provided on the surface, said field responsive particles (102a, 226a) move, at least partially in a direction normal to the surface, relative to the liquid, to alter the appearance of the composition, and
the liquid (103, 223) is disposed for fixation of said plurality of field responsive particles (102a, 226a) such that the altered appearance of the composition is preserved.

2. The composition (100, 200) according to claim 1, wherein said surface is a surface on a person.

3. The composition (100, 200) according to claim 1, wherein said surface is a surface on an animal.

4. The composition (100, 200) according to claim 1, wherein said field responsive particles (102a) are electrically charged and said external field is an electrical field.

5. The composition (100, 200) according to claim 1, wherein said field responsive particles (226a) are magnetically responsive and said external field is a magnetic field.

6. The composition (100, 200) according to claim 1, wherein said liquid (103, 223) is disposed for fixation of said plurality of field responsive particles (102a, 226a) through evaporation.

7. The composition (100, 200) according to claim 1, wherein said liquid (103, 223) comprises monomers or is polymerizable and said liquid (103, 223) is disposed for fixation of said plurality of field responsive particles (102a, 226a) through polymerisation.

8. The composition (100, 200) according to claim 1, wherein said liquid (103, 223) is disposed to initiate fixation of said plurality of field responsive particles (102a, 226a) through an action selected from a group of actions comprising heat application, chemical reaction and ultraviolet light application.

9. The composition (100, 200) according to claim 1, wherein said composition (100, 200) is a paint.

10. The composition (100, 200) according to claim 1, wherein said composition (100, 200) is a crème, said crème being removable after having been applied to a person.

11. A pattern generator for applying a field in accordance with a desired pattern to control appearance of a composition (100, 200) according to claim 1, said pattern generator selected from a group comprising a field pen, a field roller (330) and a field plate.

12. The pattern generator according to claim 11, wherein said pattern generator is additionally configured to apply said composition (100, 200) on a person.

13. The pattern generator according to claim 11, wherein the desired pattern fields are arranged in a matrix corresponding to pixels of an image.

14. An applicator for applying a composition (100, 200) according to claim 1 on a person, wherein said applicator has a hydrophobic surface for applying the composition, which is hydrophilic, or said applicator has a hydrophilic surface for applying the composition, which is hydrophobic.

15. A method for making a surface having a desired appearance, comprising the steps of:
dispensing a film forming composition on the surface comprising a plurality of field responsive particles and a liquid;
applying an external field to the such that the move, at least partially in a direction normal to the surface, relative to the liquid, to alter the appearance of the composition; and
fixating the plurality of particles.

16. The method according to claim 15, wherein the particles are electrically charged and the external field is an electrical field.

17. The method according to claim 15, wherein the particles are magnetically responsive and the external field is a magnetic field.

18. The method according to 115, wherein the step of fixating comprises evaporation of the liquid.

19. The method according to claim 15, wherein the liquid comprises monomers or is polymerizable and the step of fixating comprises polymerisation of the liquid.

20. The method according to claim 15, wherein the step of fixating comprises an action selected from a group of actions comprising heat application, chemical reaction and ultraviolet light application.

21. A coating having a desired appearance, wherein the coating is provided by a composition according to claim 1.
